(19) United States
(12) Patent Application Publication
(10) Pub. No.: US 2009/0198190 A1
(43) Pub. Date: Aug. 6, 2009

(54) Mounting Pad, an Adhesive Device Comprising Such Mounting Pad, and Method of Applying Such Devices to a Patient

(76) Inventors: Soren Jensen, Lynge (DK); Magnus W. Goransson, Malmo (SE); Grete Kornerup, Vipperod (DK); Lasse W. Mogensen, Soborg (DK)

Correspondence Address: BRINKS HOFER GILSON & LIONE, P.O. BOX 10395, CHICAGO, IL, 60610, US

(21) Appl. No.: 11/909,252

(22) PCT Filed: Mar. 20, 2006
(86) PCT No.: PCT/DK2006/050008

§ 371 Date: Aug. 21, 2008

Related U.S. Application Data
(60) Provisional application No. 60/663,804, filed on Mar. 21, 2005.

(30) Foreign Application Priority Data
Mar. 21, 2005 (DK) PA200500406

(51) Int. Cl. A61M 5/32

(52) U.S. Cl. 604/180

(57) ABSTRACT

A Mounting Pad, an Adhesive Device Comprising such Mounting Pad, and Methods of Applying such Devices to a Patient. A mounting pad having an adhesive component having an adhesive surface, said surface being provided with a release layer, said release layer having at least three projecting parts one of which is a tongue projecting along an essentially central axis of the mounting pad. The mounting pad may be used for adhesive devices, preferably injector devices for the subcutaneous introduction of the cannula of the infusion part of an infusion set into the skin of a patient. The method of applying such a device enables a reduction of the risk of damaging or contamination the adhesive surface during automatic or manual removal of the release layer when the infusion set is applied to a patient.

### BACKGROUND OF THE INVENTION

The present invention relates in its broadest aspect to mounting pads having a release layer for use in devices applied onto the skin of a patient, particularly infusion sets. The invention also relates to adhesive devices and particularly to adhesive devices including an injector device and an infusion set and a mounting pad having a release layer which can be automatically removed from the mounting pad when the infusion set is applied to a patient. The invention furthermore relates to methods of applying infusion sets to a patient.

### DESCRIPTION OF THE RELATED ART

Products in the form of an adhesive sheet material for use on the skin, comprising a release layer are well known. The release layer can be divided into several sections which can be removed manually from the adhesive device independently of each other. A product in the form of infusion sets comprising a mounting pad and a release layer are also well known. The release layer can e.g. be manually removed in one piece from the plaster before applying the infusion set on the patient.

These types of products require manual removal of the release layer before or after the infusion set is applied to the patient. In some cases when the release layer is removed manually before the infusion set is applied to the patient, the adhesive layer can fold, become contaminated or be inappropriately attached to the patient. When the release layer is removed after the infusion set has been applied to the patient the removal of the release layer can cause difficulties and discomfort when fastening the infusion set to the patient, as the infusion set is not secured to the user when the release layer is removed, although the cannula is already inserted into the patient. The release layer is often removed in one piece.

With the known products it can also be difficult to remove all of the release layer, especially in the cases where the release layer is in one piece.

A product in the form of a system for delivering adhesive strips and transparent dressings to a patient, especially very thin, adhesive-coated transparent films are known. Such a system comprises a multi-layer system, where the removal of one layer exposes an adhesive surface of the underlying layer. A support layer carrying the underlying layer can then e.g. be used for applying the underlying layer on the skin and the support layer can subsequently be removed.

It is also known to sell bandage in packages where the package constitute at least a part of the release layer. The bandage in this form can include a release layer which is fastened to the package. The bandage is applied to a wound by manually opening the package by pulling flaps and thereafter by continuing the pulling motion applying the bandage to the wound until the bandage is attached to the skin.

However, these systems are not fully automatic, and do not relieve the user of detailed and fine finger movements being problematic for people with bad dexterity. Neither does the system facilitate applying devices or parts of devices onto a patient. These systems are furthermore not rapid, which might be desirable in some instances.

Diabetes is a disease which requires constant observation of the glucose level in the patient's blood and injections of insulin. To control and facilitate the treatment some diabetics use an insulin pump connected to an infusion device for subcutaneous delivery into the patient, often through a cannula. On certain occasions, e.g. when taking a bath the pump can be disconnected from the infusion device and reconnected later while the infusion device is left in place on the patient. The subcutaneous application of the infusion device can sometimes be difficult and painful for some diabetics for which the application device according to the present invention can be helpful.

Accordingly, it is an object of the invention to provide an adhesive device which is rapid and easy to apply to a patient.

It is another object of the invention to provide an adhesive device with automatic removal of the release layer.

It is a further object of the invention to provide a rapid and easy method of applying an infusion set to a patient.

It is still a further object of the invention to provide a method of applying an infusion set to a patient without contaminating or damaging the adhering effect of the adhesive device.

### SUMMARY OF THE INVENTION

The present invention relates in one aspect to a mounting pad having an adhesive surface, said surface being provided with a release layer, said release layer having at least three projecting parts one of which defines an essentially central axis of the mounting pad.

According to a second aspect the present invention relates to an adhesive device comprising an application device and a mounting pad having an adhesive surface, said surface being provided with a release layer, wherein the mounting pad is displaceable in relation to the housing of the application device, and wherein the release layer is secured to the housing.

According to a third aspect the present invention relates to an injector device for the subcutaneous introduction of a cannula of an infusion part of an infusion set into the skin of a patient, said infusion part comprising a mounting pad having an adhesive component having an adhesive surface, said surface being provided with a release layer, said release layer having at least three projecting parts one of which is projecting along an essentially central axis of the mounting pad, said device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means, said device further comprising a pivoting member which can be swung into a position in which it embraces the needle, wherein a projecting part of the release layer is secured to the housing of the injector device.

According to a fourth aspect the present invention relates to an injector device for the subcutaneous introduction of a cannula of an infusion part of an infusion set into the skin of a patient, said infusion part comprising a mounting pad having an adhesive component having an adhesive surface, said surface being provided with a release layer, said release layer having at least three projecting parts one of which is projecting along an essentially central axis of the mounting pad, said device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means, said device further comprising a pivoting member which can be swung into a position a pivoting member which can be swung from a position in which it is placed parallel to the housing into a position in which it embraces the needle, wherein a projecting part of the release layer is secured to the housing of the injector device.

According to a fifth aspect the present invention relates to a method of applying an infusion set to a patient, comprising
providing an infusion part having a mounting pad having an adhesive component having an adhesive surface, said surface being provided with a release layer, said release layer having at least three projecting parts one of which is projecting along an essentially central axis of the mounting pad and an injector device, said injector device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means,
said device further comprising a pivoting member which can be swung into a position in which it embraces the needle,
placing the infusion part in the injector device,
setting the spring of the injector device,
securing or connecting a projecting part of the release layer to the housing of the injector device,
placing the injector device with the front part thereof in contact with the skin of a patient where the cannula is to be inserted, and
actuating the injector device
removing the first section of the release layer
pressing front part of the mounting pad against the skin.

The second and third sections of the release layer are removed together with the application device.

This is a method where the release layer is automatically removed during application. In a preferred embodiment the infusion set is delivered with the infusion part placed in the infusion set and the spring in a set condition and the projecting parts of the release layer secured to the housing of the injector device. Thus, it is only necessary to place the set at the desired site, actuate the injector device and to remove the first section of the release layer.

According to a sixth aspect the present invention relates to a method of applying an infusion set to a patient, comprising
providing an infusion part having an mounting pad having an adhesive component having an adhesive surface, said surface being provided with a release layer, said release layer being divided into three sections and having at least three projecting parts one of which is projecting along an essentially central axis of the mounting pad and an injector device, said injector device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means, said device further comprising a pivoting member which can be swung into a position in which it embraces the needle,
placing the infusion part in the injector device,
setting the spring of the injector device,
removing a second section of the release layer,
placing the injector device with the front part thereof in contact with the skin of a patient where the cannula is to be inserted
actuating the injector device, thereby applying the mounting pad and the infusion part onto the patient
removing a first section of the release layer, and
removing the third section of the release layer.

This is a method where the release layer is manually removed in steps, where one section of the release layer is first removed, thereafter the mounting pad and infusion part are applied onto the patient and finally the third and first sections of the release layer are manually removed. In a preferred embodiment of this method the infusion set is delivered with the infusion part placed in the injector device and the spring in a set condition. Then, the first step in the method will be to remove a second section of the release layer.

According to a seventh aspect the present invention relates to a kit comprising a mounting pad, an infusion part and an injector device.

The present invention relates to a mounting pad having an adhesive component having an adhesive surface, said surface being provided with a release layer, said release layer having at least three projecting parts one of which is projecting along an essentially central axis of the mounting pad. These mounting pads may be used alone as a plaster or a dressing such as a wound dressing or preferably together with devices, for applying infusion sets or parts of infusion sets, e.g. an infusion part, onto a patient.

According to a preferred embodiment the projecting parts comprising a first projecting part defining a central axis of the mounting pad and at least two secondary projecting parts are essentially parallel.

According to a preferred embodiment the secondary projecting parts are positioned symmetrical around the central axis defined by the first projecting part.

According to another preferred embodiment, the release layer is divided into two or more sections.

According to yet another preferred embodiment, the release layer is divided into at least two sections, a first section being provided with a first part projecting along the essentially central axis of the mounting pad, and two projecting parts, one on each side of first part, and a remaining section being provided with two projecting parts.

According to a further embodiment, the two projecting parts of the remaining section overlap with the two projecting parts of the first section.

According to another preferred embodiment, the remaining section of the release layer is divided into two sections, a second and a third section.

According to a further preferred embodiment, the remaining section of the release layer is divided into a second and third section along the central axis of the mounting pad.

According to yet another preferred embodiment, the remaining section is divided perpendicularly to the central axis of the mounting pad.

According to another preferred embodiment, the sections of the release layer can be removed from the mounting pad independently from each other. Preferably the mounting pad comprises a hole located along essentially a central axis of the mounting pad allowing passage of a cannula to be inserted into the skin.

According to a preferred embodiment the mounting pad has an adhesive surface being provided with a release layer, said release layer being divided into three sections perpendicularly to the central axis of the mounting pad, a first section having a first part projecting along an essentially central axis of the mounting pad and two projecting parts on each side of the first part, a second section comprising two projecting parts, and a third section, wherein the projecting parts of the of the first section are essentially parallel to and spaced apart from the first part and the projecting parts of the second section are superposed on the two projecting parts of the first section when the sections are arranged together on the adhesive surface.

According to another preferred embodiment, the mounting pad has an adhesive surface being provided with a release layer, said release layer being divided into three sections, a first section having a first part projecting along an essentially central axis of the mounting pad and two projecting parts on each side of the first part, said first section being placed on the front part of the adhesive surface between the front part of the adhesive surface and the second and third sections, the second and third sections each comprising one projecting part, said second and third sections being separated along the central axis of the mounting pad, and the projecting parts of the second and third sections of the release layer being superposed on the projecting parts of the first section.

The present invention relates in a second aspect to an adhesive device comprising an application device and a mounting pad having an adhesive surface, said surface being provided with a release layer, wherein the mounting pad is displaceable in relation to the housing of the application device during application, and wherein the release layer is secured to the housing.

The invention is based on the discovery that by securing a part of a release layer of a mounting pad to an application device the peeling of the release layer off the mounting pad may be effected automatically when the mounting pad is displaced in relation to the application device during application. This secures a clean adhesive surface of the mounting pad which results in optimal adhesion of the mounting pad to the user.

According to another embodiment the invention relates to an adhesive device, wherein at least one projecting part of the first, second or third section is secured to the housing of the application device.

According to a further embodiment the invention relates to an injector device wherein the mounting pad is longitudinally displaceable from a retracted position in relation to the application device by which displacement a spring is set, said device comprising means for retaining the mounting pad in that position, said device further comprising means for actuating the spring releasing the mounting pad. Accordingly, the actuation of the spring of the application device secures rapid application of the application device on the skin of the user, without the need for manual intervention.

This is highly relevant e.g. for diabetics which have to, or may desire to insert an infusion device or to place a subcutaneous sensor or the like on themselves. For some persons it is a troublesome and uncomfortable process to perform the skin penetration themselves and they therefore need a device which assists them in this process, thereby making the process less problematic. It is also relevant for older persons or persons with weak fingers or limited dexterity to be able to apply such devices on themselves. In those cases the adhesive devices of the present invention assist by providing an automatic and rapid application of a device on the user's body which does not require fine finger movements.

According to one embodiment of the present invention the release layer of the mounting pad is divided into two or more sections. According to another embodiment at least one projecting part of the first, second or third section is secured to the housing of the application device.

The adhesive component is preferably a skin friendly adhesive known per se, provided with a micro porous backing, one side of the component having an adhesive surface.

The adhesive surface of a mounting pad is provided with a release layer for protecting the adhesive surface until use. The release layer is preferably made from siliconized paper or from a sheet of polyethylene which can easily be removed from the adhesive surface without damaging the same.

According to a preferred embodiment of the invention the release layer provided with a non-touch feature known per se enabling an easy manipulation of the release layer without risk of touching the adhesive surface, especially in case of an embodiment for manual removal of the release layer.

According to another preferred embodiment of the invention the mounting pad of the adhesive device includes an infusion part of an infusion set.

According to yet another preferred embodiment of the invention the application device of the adhesive device is an injector device.

The adhesive device comprises an application device and an infusion set or a part of an infusion set comprising an infusion part for insertion into a patient, said infusion part comprising an mounting pad and a release layer covering at least a part of the mounting pad, said infusion set being displaceable in relation to the application device, wherein at least a part of the release layer is connected to the application device.

In a preferred embodiment the infusion part comprises a base part with a first set of guiding means, a first cannula extending from said base part and being in fluid communication with a cavity which is optionally covered with a membrane, said cavity being further adapted to receive a second cannula extending from a connector, a second set of guiding means adapted to fit with the first set of guiding means and at least two arms, said retention devices extending from an upper surface of the main surface of the base part, further comprising an injector device, said release layer being secured to the injector device.

In a third aspect the invention relates to an injector device for the subcutaneous introduction of a cannula of an infusion part of an infusion set into the skin of a patient, said infusion part comprising a mounting pad having an adhesive component having an adhesive surface, said surface being provided with a release layer, said release layer having at least three projecting parts one of which is projecting along an essentially central axis of the mounting pad, said device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means, said device further comprising a pivoting member which can be swung into a position in which it embraces the needle, wherein a projecting part of the release layer is secured to the housing of the injector device.

In a fourth aspect of the invention, the injector device is an injector device for the subcutaneous introduction of a cannula of an infusion part of an infusion set into the skin of a patient, said infusion part comprising a mounting pad having an adhesive component having an adhesive surface, said surface being provided with a release layer, said release layer having at least three projecting parts one of which is projecting along an essentially central axis of the mounting pad, said device comprising a housing, a back and longitudinally extending guiding means, a member which is longitudinally slidable within the housing and comprising a needle for insertion in the cavity of said first cannula, a spring located between the back of the housing and the longitudinally slidable member, locking means for maintaining the spring in a compressed state and release means for disengaging the locking means, said device further comprising a pivoting member which can be swung from a position in which it allows for insertion of the needle into a position in which it embraces the needle, wherein a projecting part of the release layer is secured to the housing of the injector device.

In another preferred embodiment the invention relates to an injector device, wherein the pivoting member has fixing means for temporarily fixing a projecting part of the release layer.

In yet another preferred embodiment the invention relates to an adhesive device or an injector device, comprising a mounting pad.

In a further preferred embodiment the invention relates to an injector device, wherein the first cannula extending from said base part is being in fluid communication with a cavity which is optionally covered with a membrane, said cavity further being adapted to receive a second cannula extending from a connector.

In a preferred embodiment the cannula is a soft cannula preferably a soft cannula made of a plastics material. Preferred plastics materials for the soft cannula are materials which are sufficiently flexible to bend, when the patient moves and sufficiently rigid to avoid kinking closing off the drug supply. Further the material must be compatible with medical use i.e. irritation of the skin must be kept at a minimum, being non-toxic it must not decompose in the body, etc. Thermoplastic elastomers (TPE) are a type of material which fulfils these requirements. Examples of such useful elastomers are: polyester ethers, ECDEL, styrene based TPE, olefin based TPE, urethane based TPE, ester based TPE, amid based TPE, polyolefines and silicone rubbers. In a preferred embodiment the material is selected from the group consisting of polypropylene, C-FLEX™, mixtures of C-FLEX™ and polypropylene, LUPOLEN™ 1840H, LUPOLEN™ 3020D, PELLETHANE™ 2363-75D, PELLETHANE™ 2363-55D, TECOTHANE™ and CARBOTHANE™.

In a fifth aspect the invention relates to a method of applying an infusion set to a patient, which method comprises
providing an infusion part having a mounting pad having an adhesive component having an adhesive surface, said surface being provided with a release layer, said release layer having at least three projecting parts one of which is projecting along an essentially central axis of the mounting pad and an injector device, said injector device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means, said device further comprising a pivoting member which can be swung into a position in which it embraces the needle,
placing the infusion part in the injector device,
setting the spring of the injector device,
securing or connecting a projecting part of the release layer to the housing of the injector device,
placing the injector device with the front part thereof in contact with the skin of a patient where the cannula is to be inserted, and
actuating the injector device.

This method ensures a rapid and secure application of an infusion part to a patient in an easy manner, providing an optimum placement of a cannula included in the infusion part without causing too much pain to the user. The method furthermore minimizes contamination of the adhesive surface of the mounting pad, as the release layer is removed automatically during the application.

The present invention also renders it possible to manually utilise the mounting pad of the invention together with an application device. In this manner, a part of the release layer is removed manually before the injector device is actuated, and after the adherence of the corresponding part of the mounting pad, the projections are utilised for manually pressing the pad against the skin while manually removing the remaining parts of the release layer and pressing the adhesive surface against the skin.

When the mounting pad includes a release layer which is automatically removed, the device functions accordingly. An infusion part on a mounting pad is inserted into the application device. The two projecting parts of the second and third sections of the release layer are connected to the housing of the application device. They can be secured by pressing a button through the holes of the projecting parts and corresponding holes on the application device or e.g. by welding or gluing them to the device. Thereafter first projecting part—also called the tongue—of the first section of the release layer is secured to the pivoting member of the application device. Preferably the tongue is inserted through a hole in the pivoting member, the tongue having an enlarged surface area at the end providing a locking effect. This combination of the infusion part and the mounting pad in the application device can also be the starting point for the user. Now the application device is in a locked state, as the pivoting arm is placed parallel to the housing of the application device and the longitudinal movement of said pivoting arm along the housing is hindered by the first locking means. By pulling the pivoting member of the application device and placing it in a position perpendicular to the housing said device is ready for use. Now a front end of the application device is held in contact with the skin, preferably approximately perpendicularly to the skin, and the device is thereafter activated by pressing the release means. When the device is actuated the infusion part on the mounting pad is pushed in the direction towards the skin and during this movement the second and third section of the release layer will start to peel off the mounting pad, as the two projecting parts and the tongue are secured to the application device, preferably on different sides of the housing of the device. Accordingly, the first section of the release layer and the mounting pad will move in one direction while the second and third section of the release layer will begin to move in a deviating direction, thus gradually exposing the adhesive surface of the mounting pad. When the mounting pad gets in contact with the skin of the user the adhesive surface is exposed and adheres to the skin and the cannula of the infusion part is placed subcutaneously in the patient with the help of the needle of the application device. The tongue of the first section is released from the pivoting member and the application device is lifted away from the skin. During the removal of the application device the two projecting parts of the first section are pressed down using two fingers in order to hold the mounting pad in place and to ease the pressing of the rest of the mounting pad to the skin for it to adhere correctly. The needle is removed from the cannula of the infusion part and the second and third sections of the release layer are completely removed from the mounting pad. The first section is removed by pulling the tongue. The mounting pad is now secured flat on the skin of the patient and the cannula of the infusion part is subcutaneously inserted into the patient. Finally for safety reasons the needle of the application device is destroyed by folding the pivoting member over the needle and by securing the pivoting member the third locking means.

In a preferred embodiment the pivoting member is swung from the position essentially orthogonal to a main axis of the application device, 180 degrees to a second position embracing and securing the needle said position also being essentially orthogonal to said main axis. Optionally the needle is destroyed in the process and secured in the pivoting member.

According to a preferred embodiment the invention relates to a method of applying an infusion set to a patient, comprising
providing an infusion part having a mounting pad provided with a release layer
removing the second part of the release layer
applying the mounting pad with the infusion part onto a patient
removing the third section of the release layer, and
removing the first section of the release layer.

In accordance with a sixth aspect the invention relates to a method of applying an infusion set to a patient, said method comprising
providing an infusion part having an mounting pad having an adhesive component having an adhesive surface, said surface being provided with a release layer, said release layer being divided into three sections and having at least three projecting parts one of which is projecting along an essentially central axis of the mounting pad and an injector device, said injector device comprising a housing, a back and longitudinally extending guiding means, a member which is longitudinally slidable within the housing and comprising a needle for insertion in the cavity of said cannula, a spring located between the back of the housing and the longitudinally slidable member, locking means for maintaining the spring in a compressed state and release means for disengaging the locking means, said device further comprising a pivoting member which can be swung into a position in which it embraces the needle,
placing the infusion part in the injector device,
setting the spring of the injector device,
removing a second part of the release layer,
placing the injector device with the front part thereof in contact with the skin of a patient where the cannula is to be inserted
actuating the injector device thereby applying the mounting pad with the infusion part onto the patient
removing a third section of the release layer, and
removing the first section of the release layer.

When the mounting pad includes a release layer for manual removal the device functions in a similar manner as described above until the device is activated. When the pivoting member is pulled back, the second section of the release layer is exposed and subsequently peeled off by pulling one of the projecting parts exposing the adhesive surface underneath. Now the application device is held in contact with the skin, preferably perpendicularly to the skin, and the device is thereafter activated by pressing the release means. When the device is actuated the infusion part on the mounting pad is pushed in the direction to the skin. When the mounting pad gets in contact with the skin of the user the exposed adhesive surface adheres to the skin. The cannula of the infusion part is now subcutaneously inserted into the patient with the help of the needle in the application device. The centrally projecting part of the first section is released from the pivoting member and the application device is lifted away from the skin. During the removal of the application device the two projecting parts of the first section are pressed down using the fingers in order to hold the mounting pad in place. At the same time one of the projecting parts of the third section of the release layer are pulled to remove the third section. The two projecting parts of the first section also ease the pressing of the rest of the mounting pad to the skin in order for it to adhere to in correctly. The needle is removed from the cannula of the infusion part during the removal of the application device. The first section is removed by pulling the projecting part. The mounting pad is now secured flat on the skin of the patient and the cannula of the infusion part is subcutaneously inserted into the patient. Finally for safety reasons the needle of the application device is destroyed by folding the pivoting member over the needle and by securing the pivoting member the third locking means.

The invention also relates to a kit comprising an mounting pad, in combination with an infusion part and an injector device.

### Definitions:

The expression “mounting pad” is used in the present context to designate an adhesive component having a backing layer and an adhesive layer having an adhesive surface for securing a medical device such as a plaster, a wound dressing, an ostomy appliance, or an infusion set to the skin of a patient.

The expression “release layer” is used in the present context to designate a protective layer, which protects the adhesive surface of the mounting pad before use. The release layer covers essentially the entire adhesive surface and can extend beyond the boundaries of the mounting pad forming extensions or projecting parts extending beyond the boundaries of the mounting pad. The extensions/projecting parts facilitate gripping the release layer for removal thereof or securing the same to an application device. The expression “projecting part” is used in the present context to designate a part of a release layer which extends beyond the boundary of the adhesive surface it protects. A “projecting part” can either be made of the same material and/or layer as the release layer or be a separate unit attached to the release layer.

The expression “adhesive device” is used in the present context to designate a medical device such as a plaster, a wound dressing, an ostomy appliance, or an infusion set having an adhesive surface for adhering to the skin of a patient.

The expression “application device” is used generally to designate a device for the application of an adhesive device onto the skin of a patient, or for the subcutaneous introduction of e.g. a cannula and/or a needle into the skin of a patient.

The expression “injector device” is used in the present context to designate a device for the subcutaneous introduction of the cannula of the infusion part of an infusion set into the skin of a patient.

The expression “infusion set” is used in the present context to designate a set comprising an infusion part provided with a cannula to penetrate the skin of a person and a connector for connecting the infusion part with a medical device preferably a medical delivery device such as an insulin pump. An infusion set has in its assembled form a substantially planar rear side and a relatively large width compared to its thickness, thus allowing it to lie flat on the patient's skin and thereby minimizing the discomfort of carrying the infusion set. The infusion part is placed in the patient for a longer and not specified time period while the connector can be connected and disconnected from time to time. Hereby it is possible for the patient to disconnect from the medical device, move around and at a later point re-connect to the medical device. Further it is possible to shift between different medical devices using the same infusion part thereby only one penetration of the skin is needed providing less discomfort to the patient.

The expression “infusion part” is used in the present context to designate a part of a device for subcutaneous introduction of a drug to a patient. The infusion part is provided with a cannula to penetrate the skin and is usually placed on a mounting pad for attaching the infusion part to the skin of a patient.

The expression “front part” of a release layer or an adhesive surface is used in the present context to designate a part of the release layer or adhesive surface being located nearest the site of injecting a cannula.

The expression “proximal” surface is used in the present context to designate a surface to be in contact with the user's skin, and the expression “distal surface” is used to designate a surface turned away from or in a distance to the user's skin.

### BRIEF DESCRIPTION OF THE DRAWINGS

In the following the invention will be described in further details with reference to the drawings.

FIGS. 1 A-E show top-view of an injector device of the invention illustrating the steps of application of an infusion part into the skin of a patient.

FIGS. 2 A-E show the injector device shown in FIG. 1 illustrating the steps of application of an infusion part into the skin of a patient, seen from the opposite side.

FIG. 3 shows an injector device of the invention located on the skin of a patient during application of an infusion part.

FIG. 4 shows a top view of a part of a preferred embodiment of an infusion part and a mounting pad according to the invention.

FIG. 5 shows a cross-sectional view along the line A-A of the embodiment of an infusion part according to the invention shown in FIG. 4.

FIG. 6 shows the embodiment of an infusion part according to the invention shown in FIG. 4, seen from a slanting angle.

FIG. 7 shows an embodiment of the mounting pad according to the invention seen from above (distal side).

FIG. 8 shows the embodiment of the mounting pad shown in FIG. 7, seen from the opposite side (proximal side).

FIG. 9 shows an embodiment of the first section of a release layer according to the present invention.

FIG. 10 shows another embodiment of a mounting pad according to the invention seen from above (distal side).

FIG. 11 shows the embodiment of the mounting pad shown in FIG. 10, seen from the opposite side (proximal side).

FIG. 12 shows an injector device according to one embodiment of the present invention.

### DESCRIPTION OF PREFERRED EMBODIMENTS

The invention is explained more in detail with reference to the drawings showing preferred embodiments of the invention.

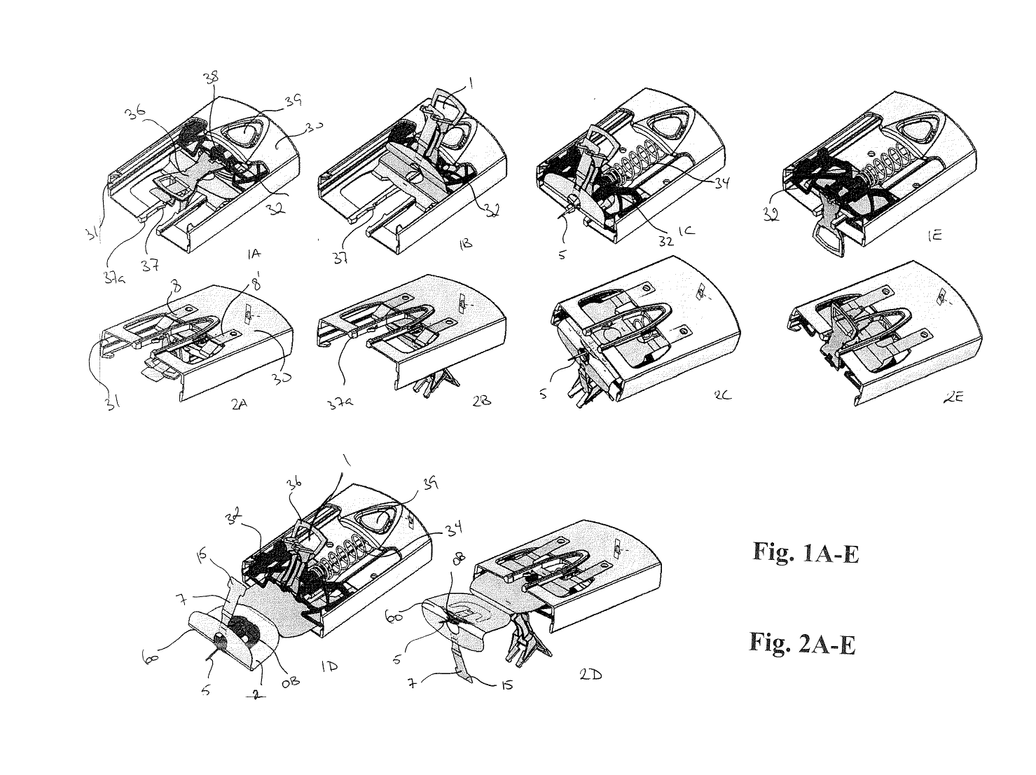

FIGS. 1 A-E show an embodiment of an injector device of the invention and illustrate the steps of application of an infusion part (0B) into a patient with the infusion part (0B) seen from above or from the distal side.

The injector device comprises a housing (30) and an infusion part (0B) provided with a mounting pad (2), which is contained in the housing (30), said injector device having a back (33) and being provided with two longitudinally extending guiding means (31) and a longitudinally slidable member (32). The device further comprises a pivoting member (36) which may engage with first means (37) for locking the pivoting member in one position where it protects the a cannula (5) of the infusion part (0B) to be introduced into a patient's skin and for preventing unintended release, and an insertion needle (35) for introducing the cannula. The pivoting member (36) has means for securing an enlarged surface area (15) of a centrally projecting part (7) of a release layer protecting an adhesive surface of the mounting pad until use. The slidable member (32) is capable of moving from a retracted position to a forward position, and is driven from the retracted position to the forward position by a spring (34). The spring is located between the slidable member (32) and the back (33) of the housing (30). The injector device further comprises second locking means (38) for locking the slidable member (32) in the retracted position when maintaining the spring in a compressed state. In this embodiment the second locking means has the form of a protrusion protruding from the slidable member (32) and being retained by a rigid part of the housing (30) placed above the slidable member (32). Also the injector comprises release means (39) for disengaging the second locking means (38), when releasing the locking means (38) the release means (39) which has the form of a button is pushed down, which pushes the locking means (38) down and then makes it possible for the locking means (38) to pass under the rigid part of the housing (30) positioned in front of the release means (39).

When the injector device is placed on the skin of a patient and the locking means is disengaged, the spring (34) drives the slidable member (32) and the infusion part (0B) to its forward position, thus introducing the cannula (5) of the infusion part into the patient by means of the needle. The release layer is provided with projections (8, 8′) being secured to the housing (30), so when the slidable member (32) moves to its forward position, the release layer is automatically removed. After the introduction of the cannula the injector device can be withdrawn leaving the needle in a free position, and the infusion part is secured to the skin using the mounting pad (2). The pivoting member (36) can then be swung into a position in which it embraces the needle and protects patients or assisting persons from being stung.

The device is shown in a first, locked state (1A) in which the device is ready for use. The device is preferably delivered ready to use and in a locked state in which projections (8, 8′) are secured to the housing (30). FIG. 1B shows the device after swinging the pivoting member (36) to a position where the pivoting member (36) is released of the first locking means (37) thereby unlocking the slidable member (32) and FIG. 1C shows the device after pressing the release means (39) and unlocking the second locking means (38) and the slidable member (32) has moved to its forward position inserting the needle and the cannula (5). FIG. 1D shows the device just before full disengagement from the infusion part (0B) and after withdrawal of the needle and FIG. 1E shows the device swinging the pivoting member into the position embracing the needle. The pivoting member may be locked in this position by third locking means (37a).

In FIG. 1B it can be seen how the second and third section of the release layer (20, 21) are peeled off the mounting pad, when the central projecting part (7) and hence first release layer is pulled back by the pivoting member (36), thereby revealing the adhesive surface (60) of the mounting pad (2).

FIGS. 2 A-E show the injector device shown in FIG. 1 illustrating the steps of application of an infusion part into the skin of a patient, seen from the proximal side.

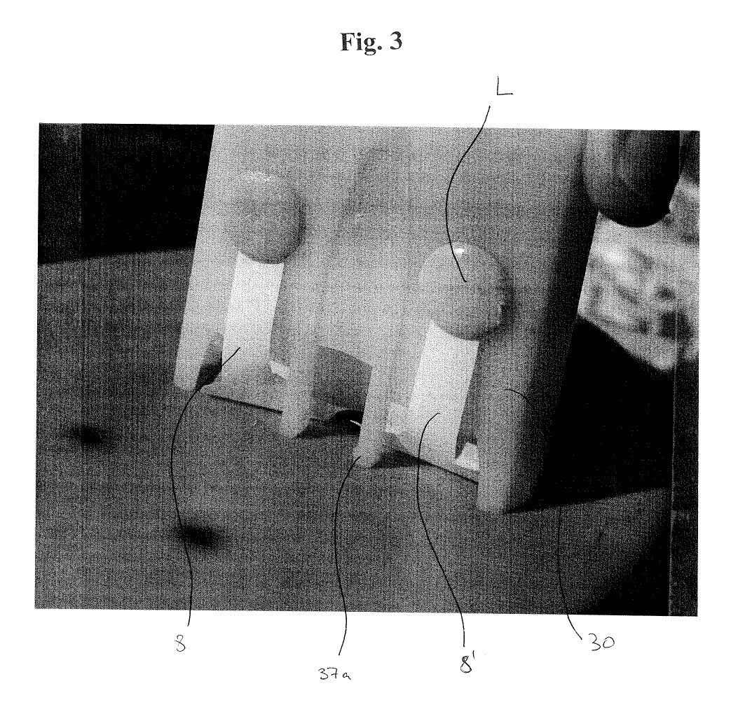

FIG. 3 shows an embodiment of an injector device of the kind shown in FIGS. 1 and 2 located on the skin of a patient during subcutaneous application of an infusion part into the patient, the device is seen from the same side as in FIG. 2A-E. The device is preferably placed perpendicularly to and in contact with the skin surface before releasing the slidable member. In FIG. 3 is shown the projections (8, 8′) and how they are secured to the housing (30) by securing means (L) which in this embodiment has the form of buttons which are pushed into holes in the housing (30). Also the third locking means (37a) for locking the pivoting member in the position in which the pivoting member embraces the needle are shown.

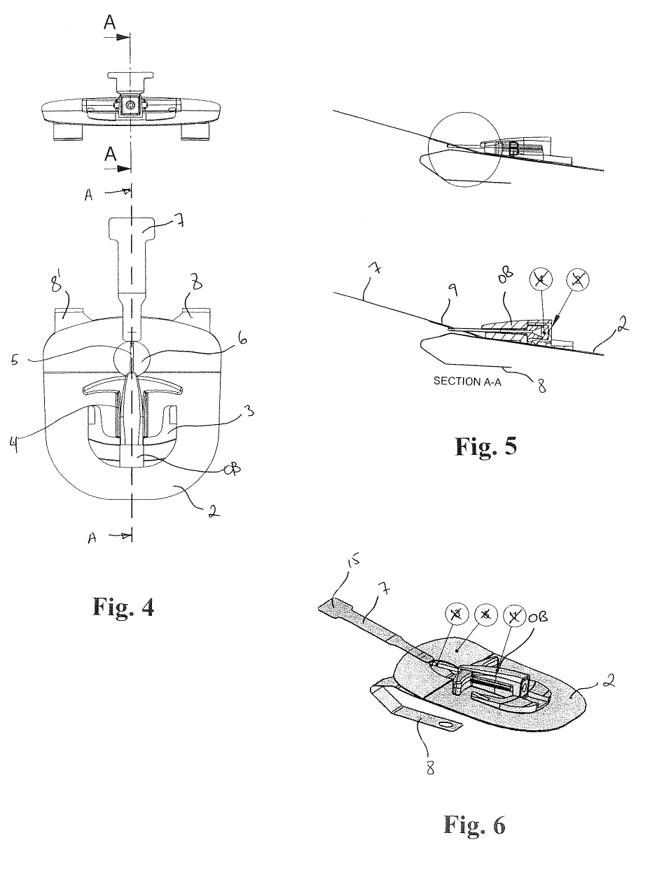

FIG. 4 shows a part of one embodiment of the invention, an infusion part (0B) and a mounting pad (2). The infusion part comprises a base part (3) having a main plane which, when the infusion set is attached to a patient, is essentially parallel with the skin of the patient. Said base part comprises a first set of guiding means (4). Mounted on the proximal surface of the infusion part is the mounting pad (2) which in this case is a plaster. A first cannula (5), preferably a soft cannula, extends from the base part and into a hole (6) in the mounting pad (2), said cannula being in fluid communication with a cavity (not shown). The cavity optionally being covered by a membrane is adapted to receive a second cannula (not shown) extending from an external connector. The mounting pad (2) furthermore has a release layer (not shown) protecting essentially the entire adhesive surface. A central projecting part (7) extends from the mounting pad (2) and defines a central axis of the mounting pad (4). The central projecting part (7) can either be made of the same layer as the release layer or be a separate unit attached to the release layer. The mounting pad (2) furthermore comprises two additional projecting parts (8, 8′) parallel to the central projecting part (7), one on each side. From this view the projecting parts (8, 8′) can not be seen in full size as they are folded under the adhesive device.

FIG. 5 is a cross-sectional view of a part of the adhesive device as shown in FIG. 1, along the line A-A. Shown are the infusion part (0B) and the mounting pad (2). The central projecting part (7) is connected to the first section of the release layer (9) and the other projecting parts (8, only on of the projecting parts is shown) are connected to the second or third section of the release layer and folded under the mounting pad (2).

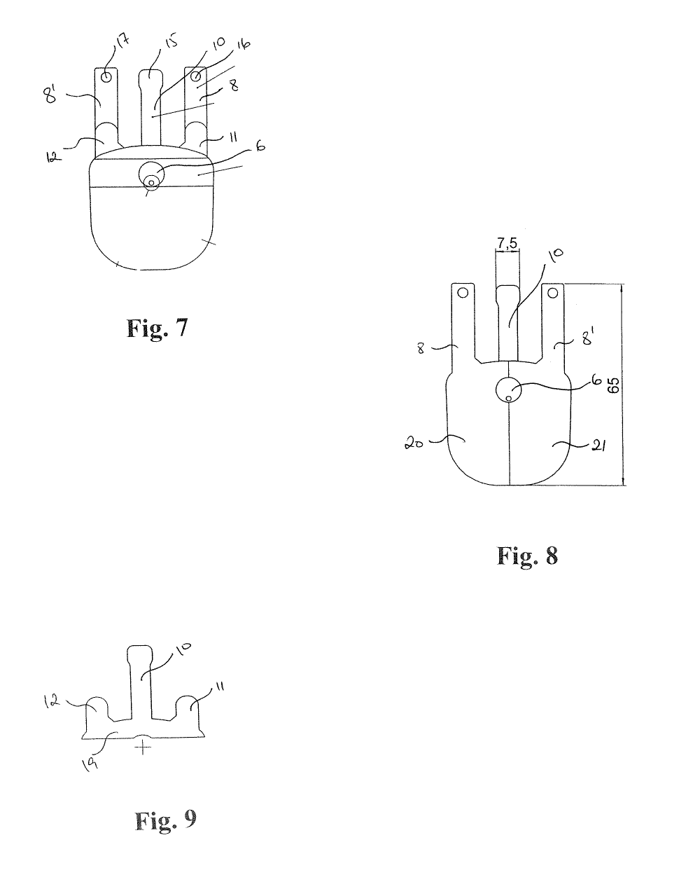

FIG. 6 shows one embodiment of a part of the adhesive device in a state, as it is when contained in the injector device. This embodiment of mounting pad is the similar to the ones shown in FIGS. 7-8. The device comprises a mounting pad (2) and an infusion part (0B). The release layer comprises two projecting parts on the second and third section of the release layer (8, only one can be seen). The two projecting parts of the first section of the release layer shown in FIGS. 7 and 8 are not included in this embodiment. The central projecting part (7) of the first section of the release layer is temporarily fastened to the injector device during application but can be released from the injector device later, e.g. manually. The central projecting part (7) is either a part of or connected to the first section of the release layer, while the other projecting parts (8) are a part of or connected to the second or third section of the release layer.

FIGS. 7-11 show the mounting pad according to the present invention. The mounting pad comprises in general an adhesive component protected with a release layer. The structure of the mounting pad shown in the figures is divided into three sections, a first, a second and a third section.

FIG. 7 shows the mounting pad in the embodiment used in FIG. 1, seen from above. The mounting pad has an adhesive surface, said surface being provided with a release layer, said release layer being divided into three sections, a first section having a tongue (10) defining an essentially central axis of the mounting pad and two projecting parts (11, 12) on each side of the tongue (10). As the first section is placed between the adhesive surface of the mounting pad and a second and a third section only the projecting parts of it can be seen from this view-point. Accordingly, the first section of the release layer is sandwiched between a part of the mounting pad substantially covering the body part of the first section of the release layer, and the corresponding parts of the second and third section of the release layer. The layered structure of the mounting pad is thus in 3 layers; primary layer being the mounting pad; secondary layer being the firs section of the release layer and the tertiary layer being partly the second section and partly the third section of the release layer. The second section comprises one projecting part (8) and a third section comprises one projecting part (8′). The second and third sections can as well only be seen by their projecting parts (11, 12) from this view-point. The second and third sections are separated along the central axis and have their projecting parts (8, 8′) arranged on top of the projecting parts (11, 12) of the first section. The projecting parts (8, 8′) have holes (16, 17) for attaching the same to the housing of an injector device, e.g. using buttons, clips or other securing means. In FIG. 8 it can be seen that besides the firs section of the second part of the release layer is divided into at least two parts, a second (20) and a third (21) section of the release layer. The second and third sections of the release layer protect the largest part of the adhesive surface of the mounting pad, which firmly secures the mounting pad to the skin of a patient.

The mounting pad has a hole for providing access e.g. for a cannula and/or a needle. This hole can have various shapes and forms.

Three projecting parts of the release layer (10, 8, 8′) provide means for securing the release layer to a housing of an injector device. The central projecting part (10) is a part of or connected to the first section of the release layer, while the other two projecting parts (8, 8′) are parts of or connected to the second and third sections of the release layer. In the shown embodiment, the central projecting part (10) is adapted to be secured to one part of the housing, preferably a pivoting member while the other two projecting parts (8, 8′) will be secured to a different part of the housing. The end of the central projecting part (10) has an enlarged surface area (15) for securing the projecting part (10) to the pivoting member (36) of an injector device.

This embodiment of the mounting pad functions accordingly when it is a part of the adhesive device according to the present invention. The injector device including the infusion part on the mounting pad is placed in close contact with the skin of the user. A pivoting member on the injector device is pulled back in order to unlock the device and to prepare the application of the infusion set, and subsequently the injector device is actuated and the infusion part is applied on the skin of the patient. As the central projecting part (10) is attached to the pivoting member and the other two projecting parts (8, 8′) of the second and third sections are secured to the other side of the housing, when the injector device is actuated, the central projecting part (10) pulls a part of the release layer in one direction while the other two projecting parts (8, 8′) pull in the opposite direction, thereby peeling the release layer off the mounting pad and exposing the adhesive surface underneath. The second and third sections remain attached to the housing while the first section can be undone from the pivoting member. Finally the first section of the release layer is removed manually by pulling the central projecting part (10).

FIG. 8 shows a top view of the mounting pad according to the invention. The embodiment is the same as described in FIG. 7 but viewed from below. The central projecting part (10), or a tongue projection, is a part of or connected to a first section of the release layer, which can not seen from this view-point, while the other two projecting parts (8, 8′) are parts of the second (20) and third (21) section of the release layer. The second (20) and third (21) sections of the release layer essentially cover a larger part of the adhesive layer than the first release layer. By separating the release layer into the second (20) and third (21) sections along the central axis of the mounting pad, it is made possible to quickly and automatically remove the release layer without influencing the movement of the needle during insertion, and avoiding oblique forces acting on the cannula.

FIG. 9 shows one embodiment of the first section (19) of the release layer which can be used in the embodiments shown in FIGS. 2, 3, 5 and 6.

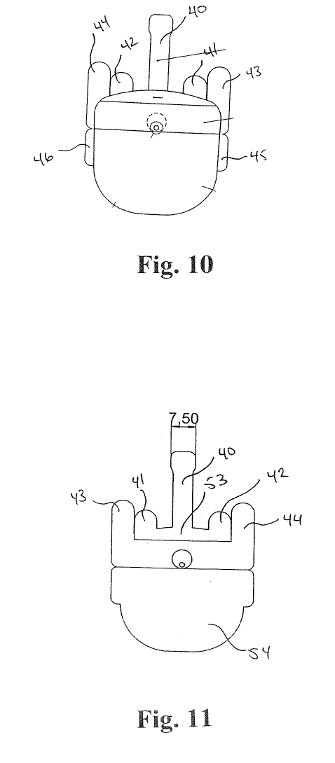

FIG. 10 shows another embodiment of the mounting pad according to the present invention, viewed from above. This embodiment is suitable for manual removal of the release layer. The mounting pad has an adhesive surface, said surface being provided with a release layer, said release layer being divided into three sections perpendicular to the central axis of the mounting pad, a first section (body part can not be seen from this view) having tongue (40) defining an essentially central axis of the mounting pad and two projecting parts (41, 42) on each side of the tongue (40), a second section (can not be seen) comprising two projecting parts (43, 44) and a third section (can not be seen from this view) comprising two projecting parts (45, 46). The projecting parts (41, 42) of the first section are adjacent to the tongue (40) and the projecting parts (43, 44) of the second section are adjacent to the two projecting parts (41, 42) of the first section when the sections are arranged together. The third section comprises two minor projecting parts (45, 46) to facilitate the removal of the third section of the release layer. From this view only the projecting parts and tongue can be seen.

This embodiment of the mounting pad functions accordingly when it is a part of the adhesive device according to the present invention. The injector device including the infusion part on the mounting pad is placed in close contact with the skin of the user. A pivoting member on the injector device is pulled back in order to unlock the device and to prepare the application of the infusion set. When the pivoting member is pulled back the second section of the release layer is accessible and subsequently removed by pulling one of the projecting parts (43, 44) thereby exposing the adhesive surface underneath. The injector device is actuated and the infusion part is applied on the skin of the patient. The tongue (40) is thereafter manually released from the pivoting member. To facilitate the removal of the second section of the release layer the user applies pressure with his/her fingers on projecting parts (41, 42) while removing the third section of the release layer by pulling one of projecting parts (45, 46) and finally the first section of the release layer is removed manually by pulling the tongue (40).

FIG. 11 shows the same embodiment of the mounting pad as shown in FIG. 5 (10), viewed from below. In this figure it is apparent that the outermost projecting parts (43, 44) are a part of the second section (52) of the release layer. Accordingly, this second section (52) can be removed separately from the first (53) and third (54) sections, e.g. prior to actuation of the injector device or when the injector device is actuated.

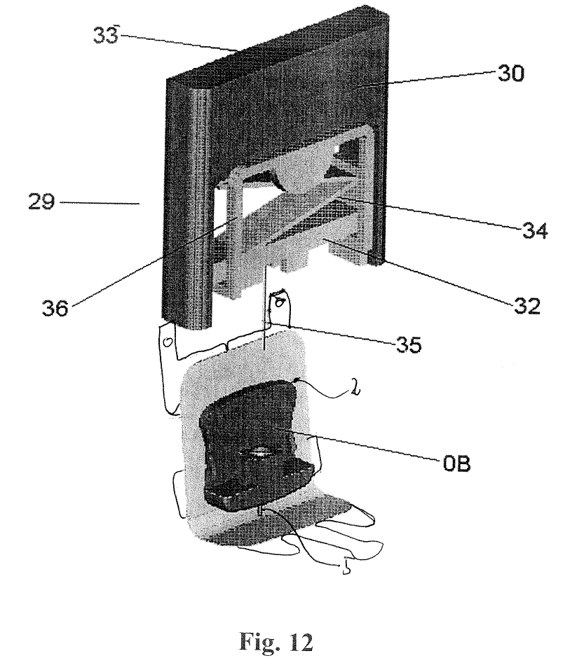

FIG. 12 shows an embodiment of an injector device (29) according to the present invention in which the injector device and an infusion part (0B) provided with a mounting pad (2) are shown in an exploded view. The injector device comprises a housing (30) having a back (33) and being provided with two longitudinally extending guiding means (31) and a longitudinally slidable member (32). The device further comprises a pivoting member (36) which may engage with means for locking the pivoting member in one position where it protects the a cannula (5) of the infusion part (0B) to be introduced into a patient's skin and a needle (35) for introducing the cannula. The slidable member is capable of moving from a retracted position to a forward position, and is driven from the retracted position to the forward position by a spring (34). The spring is located between the slidable member (32) and the back (33) of the housing (30). Optionally there is a spring support which fits with the back of the housing thereby minimizing the risk of a malfunctioning spring. The injector device further comprises locking means for (not shown) for locking the slidable member (32) in the retracted position for maintaining the spring in a compressed state and release means (not shown) for disengaging the locking means. When the locking means is disengaged, the spring (34) drives the slidable member (32) and the infusion part (0B) to its forward position, thus introducing the cannula (5) of the infusion part into the patient by means of the needle (35). After the introduction of the cannula the injector device can be withdrawn leaving the needle in a free position, and the infusion part is secured to the skin using the mounting pad (2). The pivoting member (36) can then be swung into a position in which it embraces the needle and protects patients or assisting persons from being stung.

Furthermore, the figure also schematically shows the release layer of the kind shown in FIGS. 7-9.

1. An adhesive device comprising:
an application device having a housing; and
a mounting pad having an adhesive surface, said surface being provided with a release layer, the release layer being configured to be automatically peeled away from the mounting pad when the mounting pad is displaced in relation to the application device.

2. An adhesive device according to claim 1 wherein the mounting pad is displaceable in relation to the housing of the application device, and the release layer is secured to the housing.

3. An adhesive device according to claim 1, wherein the mounting pad is longitudinally displaceable in relation to the housing of the application device, said housing comprising means for retaining the mounting pad in a displaced position set by a spring, said device further comprising means for actuating the spring and releasing the mounting pad.

4. An adhesive device according to claim 1, wherein the release layer is divided into two or more sections.

5. An adhesive device according to claim 1, wherein at least one projecting part of the release layer is secured to the housing of the application device.

6. An adhesive device according to claim 1, wherein the release layer provides a non-touch feature.

7. An adhesive device according to claim 1, wherein said release layer comprises at least three projecting parts where a first projecting part defines an essentially central axis of the mounting pad.

8. (canceled)

9. (canceled)

10. An adhesive device according to claim 7, wherein the release layer is divided into at least two sections, a first section being provided with a first projecting part and two secondary projecting parts, one secondary projecting part on each side of the first projecting part, and a remaining section being provided with two projecting parts.

11. An adhesive device according to claim 10, wherein the two projecting parts of the remaining sections overlap with the secondary two projecting parts of the first section.

12. An adhesive device according to claim 10, wherein the remaining section of the release layer is divided into two sections.

13. (canceled)

14. (canceled)

15. An adhesive device according to claim 1, wherein a first section and a remaining section of the release layer can be removed from the mounting pad independently from each other.

16. An adhesive device according to claim 7, wherein the adhesive component is a skin friendly adhesive provided with a microporous backing.

17. An adhesive device according to claim 7, wherein the release layer is made of siliconized paper.

18. An adhesive device according to claim 1, wherein said release layer comprises three sections perpendicular to the central axis of the mounting pad, a first section having a tongue projecting along an essentially central axis of the mounting pad and two secondary projecting parts on each side of the tongue, a second section comprising two projecting parts and a third section, wherein the secondary projecting parts of the first section extend essentially parallel to and spaced apart from the tongue and the projecting parts of the second section are superimposed on the two secondary projecting parts of the first section when the first and second sections are arranged together on the adhesive surface.

19. An adhesive device according to claim 1, wherein the release layer being divided into three sections, a first section having a tongue projecting along an essentially central axis of the mounting pad and two secondary projecting parts on each side of the tongue, said first section being placed on a front part of the adhesive surface between the front part of the adhesive surface and the second and third sections, the second and third sections each comprising one projecting part, said second and third sections being separated along the central axis of the mounting pad, and the projecting parts of the second and third sections of the release layer being superimposed on the secondary projecting parts of the first section.

20. An adhesive device comprising:
an injector device for the subcutaneous introduction of a first cannula of an infusion part of an infusion set into the skin of a patient, said infusion part comprising:
a mounting pad having an adhesive component having an adhesive surface, said surface being provided with a release layer, said release layer having at least three separate projecting parts,
said injector device comprising a housing, a back and longitudinally extending guiding means, a member which is longitudinally slidable within the housing and comprising an insertion needle, a spring located between the back of the housing and the longitudinally slidable member, locking means for maintaining the spring in a compressed state and release means for disengaging the locking means, said device further comprising a pivoting member which can be swung into a position in which it embraces the needle,
wherein at least one projecting part of the release layer is secured to the housing of the injector device.

21. (canceled)

22. An adhesive device according to claim 20, wherein the pivoting member comprises a securing member for releasably securing a projecting part of the release layer to the pivoting member.

23. (canceled)

24. An adhesive device according to claim 20, wherein the first cannula extending from said base part is in fluid communication with a cavity which is optionally covered with a membrane, said cavity being adapted to receive a second cannula extending from a connector.

25. A method for preparing an infusion for application, comprising
providing an infusion part comprising an adhesive device comprising an application device having a housing and a mounting pad having an adhesive component having an adhesive surface, said surface being provided with a release layer, said release layer having at least three projecting parts where a first projecting part is projecting along an essentially central axis of the mounting pad and an injector device, said injector device comprising a housing, a back and longitudinally extending guiding means, a member which is longitudinally slidable within the housing and comprising a needle for insertion in the cavity of a cannula, a spring located between the back of the housing and the longitudinally slidable member, locking means for maintaining the spring in a compressed state and release means for disengaging the locking means, said device further comprising a pivoting member which can be swung into a position in which it embraces the needle,
placing the infusion part in the injector device,
setting the spring of the injector device, and
securing or connecting a projecting part of the release layer to the housing of the injector device.

26. (canceled)

27. A kit comprising an adhesive device according to claim 1, in combination with an infusion part and an injector device.

28. (canceled)

29. An adhesive device according to claim 28, wherein the secondary projecting parts are positioned symmetrically around the central axis defined by the first projecting part.

30. A method for mounting an infusion part to a patient's skin, the method comprising:
placing the infusion part into an injector device, the infusion part comprising a mounting pad including an adhesive surface, at least a portion of the adhesive surface being covered by a release layer comprising a first projecting part and a second projecting part;
connecting the first projecting part to a first portion of the injector device and connecting the second connecting part to a second portion of the injector device so that first projecting part moves in a first direction and the second projecting part moves in a second direction when the injector device is activated to move towards the patient's skin;
activating the injector device to move a portion of the infusion part towards the patient's skin and to move the infusion part relative to the injector device; and
exposing at least a portion of the adhesive surface during said relative movement.

31. The method of claims 30 wherein exposing at least a portion of the adhesive surface further comprises peeling off the first and second projecting parts in different directions.

32. The method of claim 30 wherein connecting the first connecting part to a pivoting member of the injection device.

33. The method of claim 30 wherein at least one of the projecting parts of the release layer is retained by the injection device apart for the infusion part upon mounting the infusion part to the patient's skin.
